(19) United States
(12) Patent Application Publication
(10) Pub. No.: US 2021/0068659 A1
(43) Pub. Date: Mar. 11, 2021

(54) Use of Octafluorocyclobutane for Lung Imaging

(71) Applicant: Lakehead University, Thunder Bay (CA)

(72) Inventors: Mitchell Albert, Thunder Bay (CA); Yurii Shepelytskyi, Thunder Bay (CA); Francis Hane, Thunder Bay (CA); Tao Li, Thunder Bay (CA)

(21) Appl. No.: 17/005,554

(22) Filed: Aug. 28, 2020

Related U.S. Application Data
(60) Provisional application No. 62/897,517, filed on Sep. 9, 2019.

(51) Int. Cl. A61B 5/00; A61B 5/055; A61K 49/18

(52) U.S. Cl. CPC A61B 5/004; A61K 49/1815; A61B 5/055

(57) ABSTRACT

Described herein is a new inert fluorinated gas which can be used for fluorine-19 (¹⁹F) magnetic resonance imaging (MRI) of the lungs. Specifically, this method uses 20-79% octafluorocyclobutane (OFCB) premixed with at least 21% oxygen to acquire for example static ventilation images of the lungs, dynamic multiple breathing images of the lungs, apparent diffusion coefficient measurements (ADC) of OFCB in the lungs, and ventilation-perfusion ratio (V/Q) mapping.

### PRIOR APPLICATION INFORMATION

The instant application claims the benefit of U.S. Provisional Patent Application Ser. No. 62/897,517, filed Sep. 9, 2019, and entitled “Use of Octafluorocyclobutane for lung imaging”, the entire contents of which are incorporated herein by reference for all purposes.

### BACKGROUND OF THE INVENTION

¹⁹F lung MRI is a novel imaging technique which is currently under development. Despite low image quality (fuzzy image, low signal to noise ratio), ¹⁹F MRI is used for detection of pulmonary diseases based on analysis of localized or regional lung parameters. Currently, there are two gases which are the most commonly used for lung imaging: perfluoropropane (PEP) and sulfur hexafluoride (SF6). PFP is most commonly used for human inhalations. The main limitation of ¹⁹F lung MRI is the short relaxation times of PFP and SF which leads to fast MRI signal decay which in turn limits the imaging time.

U.S. Pat. No. 9,724,015 teaches systems and methods for generating MRI images of the lungs and/or airways of a subject using a medical grade gas mixture comprises between about 20-79% inert perfluorinated gas, specifically, PFP and SF6, with PFP being preferred because of its longer T2 relaxation time, and at least 21% oxygen gas. The images are generated using acquired ¹⁹F magnetic resonance image (MRI) signal data associated with the perfluorinated gas and oxygen mixture. U.S. Pat. No. 9,724,015 is incorporated herein by reference for all purposes, particularly for disclosures on MRI lung imaging techniques.

### SUMMARY OF THE INVENTION

According to a first aspect of the invention, there is provided a method for generating a magnetic resonance image of lungs of a patient comprising:

the patient inhaling an effective amount of a gaseous mixture comprising 20-79% octafluorocyclobutane and at least 21% O₂;

generating a magnetic resonance image of the lungs of the patient while at least some of the gaseous mixture is present in the lungs of the patient; and

displaying the image.

According to another aspect of the invention, there is provided a method for generating lung ventilation information of a patient comprising:

the patient inhaling an effective amount of a gaseous mixture comprising 20-79% octafluorocyclobutane and at least 21% O₂;

generating a magnetic resonance image of the lungs of the patient while at least some of the gaseous mixture is present in the lungs of the patient;

displaying the image; and

analyzing the image for regions of low ventilation.

### BRIEF DESCRIPTION OF THE DRAWINGS

FIG. 1. (A) The axial projection image of the healthy rat lungs acquired during a single breath-hold using 79% of OFCB premixed with 21% of O₂. (B) The same image acquired with 79% of PFP. The signal-to-noise ratio (SNR) of (A) is 18% higher than the SNR of (B).

FIG. 2. (A) The axial projection image of the healthy rat lungs acquired during 3 minutes of continuous breathing using 79% of OFCB premixed with 21% of O₂. (B) The same image acquired with 79% of PFP. The SNR of (A) is 17% higher than SNR of (B)

FIG. 3. Structure of octafluorocyclobutane (OFCB).

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein is a new inert fluorinated gas for use for fluorine-19 (¹⁹F) magnetic resonance imaging (MRI) of the lungs. Specifically, this method uses a gaseous mixture comprising 20-79% octafluorocyclobutane (OFCB) premixed with at least 21% oxygen to acquire for example static ventilation images of the lungs and/or dynamic multiple breathing images of the lungs, which can be used for calculating apparent diffusion coefficient measurements (ADC) of OFCB in the lungs, and ventilation-perfusion ratio (V/Q) mapping. As discussed herein, the procedures for the acquisition of images of the lungs and for gas delivery are the same as those taught in the prior art and known to those of skill in the art, but use of OFCB provides improved scan quality and safety, for example, up to 19% better image quality and 60% safer compared to PFP.

OFCB was not used previously for MRI lung imaging due to several reasons. Because the compound had not been as well studied as PFP and SF6, it was not clear if the smaller number of signal averages would be enough to produce a higher signal compared for example to PFP. Furthermore, OFCB had no history of use in animals. Consequently, the advantages of OFCB compared to PFP and SF6 were very surprising.

The relaxation times for OFCB-O₂ mixture In Vivo should be measured experimentally. We measured the relaxation times for pure OFCB and OFCB-O₂ mixture in vitro.

While we were not first who measured the relaxation times for pure OFCB, to our knowledge, we are the first who characterized the OFCB-O₂ breathing mixture.

Specifically, one advantage of OFCB compared to the other perfluorinated gases is the greater number of fluorine per molecule (eight nuclei). That is, the OFCB molecule is a square-shaped, symmetrical molecule, as shown in FIG. 3. Generally speaking, the more equivalent nuclei per molecule, the stronger the signal. OFCB is the heaviest perfluorocarbon, and is the last perfluorinated substance which is naturally in a gaseous state at room temperature. Because of this geometry, all eight fluorenes contribute to the signal.

OFCB, when inhaled into the lungs, distributes among the lung space in a natural way (similarly to air), allowing for acquisition of a lung image. The gas produces positive contrast wherein the bright areas on the image comes from the places where there is a gas. During the scanning procedure, a desirable image can be created, depending on the pulse sequence. For example, it could be either a 2D multislice image or a 3D multislice image. If there is any obstruction of airways due to, for example, pulmonary diseases, OFCB either will not be able to reach the obstructed region or the amount of gas in the obstructed region will be significantly lower than elsewhere in the lungs which can be seen in the image. Thus, areas of low contrast are indicative of ventilation defects.

Furthermore, because of self-diffusion, it is possible to measure the diffusion tensor, which are known to be strongly correlated with alveolar size.

Finally, because the OFCB is premixed with oxygen prior to inhalation, the signal intensity inside the lungs strongly depends on the local partial pressure of oxygen. This allows us to measure local V/Q ratio and do a V/Q map which can also be indicative of a variety of lung diseases, thereby allowing for early diagnosis. As will be apparent to one of skill in the art and as discussed herein, the vast majority of lung diseases can be diagnosed using a V/Q map.

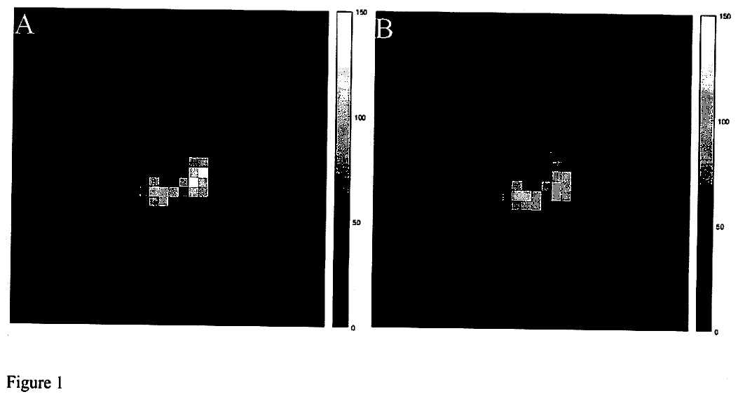

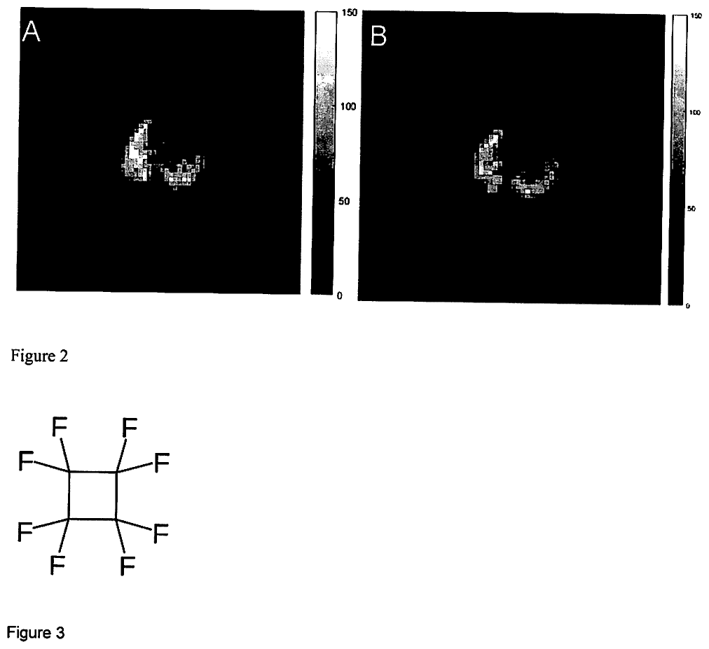

As shown in FIG. 1 and FIG. 2 and as discussed herein, we have obtained results using this approach that showed better image quality using OFCB compared to the most commonly used perfluorinated gas, PFP gas.

PFP is a quasilinear molecule (CF3-CF2-CF3) and the differences in the local magnetic field caused by differences between the CF3 and CF2 groups in the PFP molecule causes a “spurious” signal which produces a second image of the lungs. This additional image is dimmer compared to the first one and the position of this second image depends on the receiving bandwidth. This second image is called a Chemical Shift Artifact (CSA). The problem is that it can overlap with the main image, resulting in an image that is blurred and distorted and which can impair accurate diagnosis. For example, a region of the lungs showing a low signal may be interpreted as an area of poor ventilation but one that can still be considered a ventilation region when in fact, the low signal is caused by the CSA and there is no ventilation in that region of the lungs. The end result will be a lower ventilation defect percentage and incorrect ADC and V/Q values for this region. As a result, a patient with early stage COPD/asthma/IPF may be mis-diagnosed as healthy.

Generally speaking, overlapping of the main image with the CSA results in underestimation of lung disease severity. Consequently, MRI acquisitions with PFP require suppression of the signal produced by the CF2 group which makes the scanning procedure more complicated.

In contrast, as discussed above, the OFCB molecule contains 4 magnetically equivalent CF2 groups. Consequently, all ¹⁹F nuclei contribute to the MRI signal of the same frequency, resulting in a single spectral peak. Furthermore, there is no need to suppress any signal during the OFCB MRI scan. Also, the absence of additional signal means that OFCB scans are more independent of the receiving bandwidth.

Furthermore, OFCB also has a long spin-spin relaxation time. Physically, it means that the OFCB signal takes longer to decay compared to the PFP signal. This means that during the receiving time period, the amount of signal from OFCB will be higher compared to the amount of signal from PFP over the same time period.

While the spin-lattice relaxation time is 60% longer for OFCB compared to PFP, which limits the potential number of signal averages, the longer spin-lattice relaxation means that there are a smaller number of radio pulses with OFCB. This means that the Specific Absorption Rate (SAR) is lower for OFCB-O₂ scans. As will be appreciated by one of skill in the art, this parameter shows the amount of energy absorbed by tissue and, therefore, indicates the level of tissue heating caused by MRI pulses. The lower SAR value of OFCB scans means that ¹⁹F OFCB lung MRI is safer for patients than MRI scans acquired using other inert fluorinated gases.

After theoretical calculations, which take into account unequal number of signal averages, OFCB scans should have 19% higher signal-to-noise ratio compared to PFP scans. Specifically, for any possible scan parameters using a gradient echo pulse sequence, the SNR of OFCB-O₂ image should be roughly 19% better than the corresponding PFP-O₂ image for the same imaging time. The images shown in FIG. 1 and FIG. 2 show agreement with these theoretical calculations. In practical terms, because the signal to noise ratio of OFCB is higher, the OFCB scan provides more accurate numbers for ventilation defect percentage, ADC and V/Q measurements, and therefore can be used to detect lung abnormalities earlier, as discussed herein.

Specifically, OFCB can be used to detect lung diseases earlier and with greater accuracy compared to PFP, due to the higher SNR ratio, as discussed above. Usually, to calculate the ventilation defect percentage, digital clusterization is done. The main problem is distinguishing between the pixels with poor ventilation and the pixels with no ventilation which as discussed above is complicated by the CSA when using PFP. The higher signal level of OFCB simplifies this task and allows for more accurate calculation of the ventilation parameters. This applies to the ADC and V/Q measurements as well.

In summary, due to the symmetry of OFCB, all ¹⁹F nuclei of OFCB are chemically equivalent and all contribute equally to the MRI signal. The longer relaxation times of OFCB (shown in Table 1) mean slower decay times compared to other inert fluorinated gases. Together, these make OFCB an ideal inhalation agent for dynamic multiple breathing imaging as well as for static ventilation imaging.

As discussed above, ADC can be used to determine the alveolar size. This is a very important physiological parameter which can be used for direct diagnostics of asthma and pulmonary fibrosis. Specifically, if the ADC values in a patient are smaller than the values measured in healthy individuals, their alveolar sizes are smaller and there is some lung obstruction. As will be known to those of skill in the art, ADC values depend on gas type, meaning that the comparison of a given patient should be with OFCB ADC measured in healthy volunteers.

V/Q ratio provides regional information on gas exchange within the lungs. Low V/Q ratio (low perfusion) in a given region of the lungs indicates low partial pressure of the oxygen in that region. Low V/Q ratio usually is indicative of for example asthma, chronic bronchitis, and/or hepatopulmonary syndrome.

A high V/Q ratio in a given region indicates decreased partial pressure of carbon dioxide and increased pressure of oxygen. As such, a high V/Q ratio indicates that peripheral oxygen saturation is lower than normal, leading to tachypnea and dyspnea. High V/Q ratio is associated with emphysema and pulmonary embolism.

As will be apparent to those of skill in the art, Fluorine-19 MRI of the lungs can be used for a variety of purposes in addition to diagnosis. For example, ¹⁹F-MRI can be used to monitor treatment progress. Specifically, a lot of important regional parameters can be obtained from lung images which can in turn be used to determine regional function as well as regional changes in lung tissues over time. For example, for a patient diagnosed with a pulmonary disease, lung MRI could be taken over time to monitor changes in lung function as a means to determine treatment efficacy. Furthermore, ¹⁹F lung MRI is a powerful tool to monitor the effect of experimental compounds of interest on lung function. As will be appreciated by one of skill in the art, the greater accuracy, sensitivity and safety of OFCB means that this compound is ideal for this type of monitoring.

For example, OFCB can be used for sequential breath-hold images or time gated images to identify for example wash-in and wash-out information, which can be used to determine the severity of any ventilation defects.

In other embodiments of the invention, OFCB can also be used to obtain data to identify ventilation and/or perfusion variations (defects or increases) before and/or after administration of a potentially physiologically active substance to a patient, for example, a human or a test animal, to evaluate the effect of the potentially physiologically active substance or drug on the lungs. As will be appreciated by one of skill in the art, in some embodiments of the invention, this data may be obtained several times during the duration of a dosage regimen or schedule to determine efficacy of the drug on the lungs over time.

For example, as discussed herein and as will be apparent to one of skill in the art, OFCB-O₂ gas may be used to generate static and/or dynamic in vivo ¹⁹F MRI images of the lungs. For example, the OFCB-O₂ gas mixture comprising 20-79% OFCB, for example, 40-79% OFCB, and at least 21% O₂ may be administered to a patient for generating one or more MRI images of the lungs, which can be displayed and analyzed for a variety of purposes, as discussed herein.

According to an aspect of the invention, there is provided a method for generating a magnetic resonance image of lungs of a patient comprising:

ventilating the patient with an effective amount of a gaseous mixture comprising 20-79% octafluorocyclobutane and at least 21% O₂;

generating a magnetic resonance image of the lungs of the patient while at least some of the gaseous mixture is present in the lungs of the patient; and

displaying the image.

As will be apparent to one of skill in the art, the number of images depends on the imaging purpose. For example, multiple slice imaging can be used for ventilation defect percentage calculation, ventilation volume calculation and the like. To get the V/Q map, we can either conduct a T1 measurement (using inversion recovery) or acquire the same image twice using a different percent of gas (for example, 79:21 OFCB:O₂ and 30%:70% OFCB:O₂ mixtures).

In some embodiments of the invention, the gaseous mixture may comprise 40-79% octafluorocyclobutane.

As discussed herein, following a suitable time period, the method may further comprise generating a second magnetic resonance image of the lungs of the patient while at least some of the gaseous mixture is present in the lungs of the patient; and comparing the second magnetic resonance image to the magnetic resonance image.

As will appreciated by one of skill in the art, the “suitable period of time” will depend on what is being examined. For example, if generating a series of free breathing images, the period of time will be very short, for example, on the order of minutes; however, if changes in lung function over time are being examined, the “suitable period of time” may be on the order of weeks or months or longer.

As used herein, an “effective amount” is an amount that is sufficient for a suitable MRI image to be generated.

According to another aspect of the invention, there is provided a method for generating lung ventilation information of a patient comprising:

ventilating the patient with an effective amount of a gaseous mixture comprising 20-79% octafluorocyclobutane and at least 21% O₂;

generating a magnetic resonance image of the lungs of the patient while at least some of the gaseous mixture is present in the lungs of the patient;

displaying the image; and

analyzing the image for regions of low ventilation.

In some embodiments, the gas mixture may include a third gas which may include an anesthetic gas or a suitable support gas.

The gas mixture may be supplied at low pressure so that the dense gas component remains in the gaseous state under normal operating conditions of about room temperature.

For example, the patient may inhale a quantity of the OFCB-O₂ gas mixture into the pulmonary region, for example, the lungs and trachea. After inhalation, the patient can hold his/her breath for a predetermined time period, for example, 5-20 seconds which can be described as “breath-hold” delivery. In other embodiments, the patient may breathe the OFCB-O₂ gas mixture freely.

For example, a series of free breathing images may be taken, providing information on temporal and special distribution of the OFCB in the lung space and lungs of the patient to provide ventilation image data over at least one respiratory cycle, static ventilation images of the lungs, dynamic multiple breathing images of the lungs, apparent diffusion coefficient measurements (ADC) of OFCB in the lungs, and ventilation-perfusion ratio (V/Q) mapping.

For example, this ventilation image data can be used to determine a ventilation defect index for each of the right and left lungs or regions thereof or to generate a ventilation defect index map of the lungs which may show a spatial distribution of ventilation regions of the lungs; a ventilation pattern of the lungs; at least one histogram associated with wash-in and/or wash-out of the OFCB mixture; a regional ventilation defect model; gas trapping images; and a pattern depicting gas exchange to capillary blood flow.

The invention will now be further explained and/or elucidated by way of examples, although the invention is not necessarily limited to the examples.

Prior to MRI imaging the animal was anaesthetized using 2% of isoflurane until their corneal reflex became absent. Once the rats were anaesthetized, a tail vein catheter was placed and an intravenous (IV) infusion of propofol was started (45 mg/kg/hr). A midline incision was made in the neck of the rat and the trachea localized. A 1 mm semi-circumscribed incision was made in the trachea, and an endotracheal catheter was inserted into the trachea. The neck was sutured closed. The endotracheal tube was connected to a custom-made ventilator and the rats were placed on 79:21 OFCB-O₂/PFP-O₂ mixtures at 60 breaths per minute with a tidal volume of 5 mL. The single breath-hold of 11 s was initiated and the imaging was started simultaneously.

To acquire the images showed in FIG. 1, the following parameters were used: FOV=100×100 mm², 32×32 acquisition matrix, TE=0.63 ms, FA=70°, slice thickness=300 mm, bandwidth=436 Hz/pixel. The repetition times was set up to be equal to measured T₁ of the breathing mixtures. To keep the scan time equal to the breath-hold duration, the number of signal averages (NSA) were equal to 16 and 24 for OFCB and PFP breathing mixtures respectively.

To acquire the images shown in FIG. 2, no breath-hold was applied. The animal was breathing the gas mixture continuously for 3 minutes. The following imaging parameters were used: FOV=100×100 mm², 64×64 acquisition matrix, TE=0.95 ms, slice thickness of 300 mm, bandwidth=246 Hz/pixel, FA=90°. The NSA for OFCB scan was equal to 29, whereas the PFP NSA was equal to 41. The TR for OFCB scan was equal to 20 ms. The TR for PFP scan was changed to 12.6 ms.

While the preferred embodiments of the invention have been described above, it will be recognized and understood that various modifications may be made therein, and the appended claims are intended to cover all such modifications which may fall within the spirit and scope of the invention.

### REFERENCES

Couch M J, et. al. 19F MRI of the lungs Using Inert Fluorinated Gases: Challenges and New Development. J. Magn. Reson. Imag. 2016 (49), 343-354.
Couch M J, et. al. Inert fluorinated gas MRI: a new pulmonary imaging modality. NMR in Biomed. 2014 (27), 1525-1534.
Halaweish A, et. al. Perfluoropropane Gas as a Magnetic Resonance Lung Imaging Contrast Agent in Humans. Chest. 2013 (144), 1300-1310.
Gu W, et. at Dynamic 19F-MRI of Pulmonary Ventilation Using Sulfur Hexafluoride (SFs) Gas. Magn. Reson. Med. 2001 (45), 605-613.
Couch M J, et. al. Pulminary Ultrashort Echo Time 19F MR Imaging with Inhaled Fluorinated Gas Mixtures in Healthy Volunteers: Feasibility. Radiology. 2013 (269), 903-909.
Kruger S, et. al. Functional Imaging of the lungs with Gas Agents. J. Magn. Reson./mag. 2016 (43), 295-315.
Wolf U, et. at Visualization of inert gas wash-out during high-frequency oscillatory ventilation using fluorinate-19 MRI. Magn. Reson. Med. 2010(64), 1479-1483.
Friedrich J, et. al. 19F-MRI: Flow Measurement of Fluorinated Gases During High Frequency Oscillatory Ventilation. Proc. Intl. Soc. Mag. Reson. Med. 2011 (19), 3498.

TABLE 1
Measured relaxation parameters for OFCB and PFP

| | T1, ms | T2′, ms |
|---|---|---|
| OFCB | 27.9 ± 0.8 | 10.5 ± 1.8 |
| OFCB-O2 | 19.6 ± 0.3 | 8.6 ± 0.5 |
| PFP | 18.6 ± 0.4 | 6.26 ± 0.27 |
| PFP-O2 | 12.2 ± 0.6 | 5.4 ± 0.3 |

1. A method for generating a magnetic resonance image of lungs of a patient comprising:
the patient inhaling an effective amount of a gaseous mixture comprising 20-79% octafluorocyclobutane and at least 21% O₂;
generating a magnetic resonance image of the lungs of the patient while at least some of the gaseous mixture is present in the lungs of the patient; and
displaying the image.

2. The method according to claim 1 wherein the gaseous mixture comprises 40-79% octafluorocyclobutane.

3. The method according to claim 1 wherein following a suitable time period, generating a second magnetic resonance image of the lungs of the patient while at least some of the gaseous mixture is present in the lungs of the patient; and comparing the second magnetic resonance image to the magnetic resonance image.

4. A method for generating lung ventilation information of a patient comprising:
the patient inhaling an effective amount of a gaseous mixture comprising 20-79% octafluorocyclobutane and at least 21% O₂;
generating a magnetic resonance image of the lungs of the patient while at least some of the gaseous mixture is present in the lungs of the patient;
displaying the image; and
analyzing the image for regions of low ventilation.

5. The method according to claim 4 wherein the gaseous mixture comprises 40-79% octafluorocyclobutane.

6. The method according to claim 4 wherein following a suitable time period, generating a second magnetic resonance image of the lungs of the patient while at least some of the gaseous mixture is present in the lungs of the patient; and comparing the second magnetic resonance image to the magnetic resonance image.
